(19) United States
(12) Patent Application Publication
(10) Pub. No.: US 2023/0180893 A1
(43) Pub. Date: Jun. 15, 2023

(54) INSOLE AND STIMULATION METHOD

(71) Applicant: FEETME, Paris (FR)

(72) Inventors: Alexis MATHIEU, Paris (FR); Andrey MOSTOVOV, Aubervilliers (FR); Damien JACOBS, Paris (FR)

(73) Assignee: FEETME, Paris (FR)

(21) Appl. No.: 17/925,398

(22) PCT Filed: May 20, 2020
(86) PCT No.: PCT/FR2020/050847

§ 371 Date: Nov. 15, 2022

(51) Int. Cl. A43B 17/00; A43B 3/34

(52) U.S. Cl. CPC A43B 17/006; A43B 3/34

(57) ABSTRACT

An insole configured to be disposed in a shoe worn by a person, in particular during a walking period of the person. The insole includes a force and/or pressure measurement unit configured to acquire at least one representative measurement of the force and/or pressure exerted on the insole by the person, a motion measurement acquisition unit configured to acquire at least one measure representative of a motion of the person’s foot, and a processing unit configured to receive the force and/or pressure measurement and the motion measurement, to compute at least one walking parameter and to control the emission of stimulation at a walking moment according to the value of the walking parameter.

### FIELD OF INVENTION

The invention relates generally to the field of stimulation to reinforce the capabilities and quality of walking of a person.

The invention therefore relates, firstly, to a shoe insole for the stimulation of a person, and secondly, to a method of stimulation of a person wherein said person wears such an insole and a system comprising an integrated or associated stimulation unit.

### BACKGROUND OF INVENTION

Devices for stimulating a person are thus known, in particular in order to improve his walking capacities.

For example, the document US20090240171 describes a device for analyzing the asymmetry of walking between left and right feet of a person by measuring and comparing the stance time or phases while walking. A sensory response is provided to the person when a difference between these measured times or phases exceeds a predetermined threshold.

### Technical Problem

However, such devices have drawbacks.

Indeed, these are often cumbersome and not very conducive for being used in a daily context.

Furthermore, although likely to have a satisfactory temporary effectiveness, such devices do not allow to have a prolonged effect since they are used during a relatively short period of time. Indeed, these devices are most often used during a specific session in a care structure, in the hospital or other.

Moreover, such devices are difficult to customize. However, the same stimulation is not necessarily suitable for all people, given the variability of morphologies or the response to stimulations between people. In addition, it is often necessary to check whether the stimulation is suitable for the person when walking and not in a static situation. The implementation of these devices therefore requires the presence of trained medical staff to determine and adapt the stimulation to the person, which further complicates their daily adoption and ease of use.

In addition, the stimulation of such devices is often repetitive and may see the diminution of its effect over time, in particular because of a well-known phenomenon of habituation or addiction of the person to any external stimulation, so that it is necessary to calibrate these devices at regular intervals in time so that they work satisfactorily over time.

Finally, such devices can be painful to wear by the person because it is not comfortable to use over a long period of time.

There is thus a need for a stimulation device that is simple to use, smart, accessible to non-medically trained personnel, space-saving and comfortable so that it can be used in a context of daily life, that significantly enhances a person’s walking abilities and quality, that is easily adaptable to the person so that it can be used without complex modification by a variety of people and in a wide variety of everyday situations (sloping terrain, stairs, etc.) or type of walk, that is simple and less expensive to manufacture to ensure its accessibility to the general public.

### SUMMARY

For this purpose, the invention has, as its first object, an insole adapted to be arranged in a shoe worn by a person, in particular during a walking period of the person, the insole comprising:
a force and/or pressure measurement unit adapted to acquire at least one measure representative of the force and/or pressure exerted on the insole by the person,
a motion measurement acquisition unit adapted to acquire at least one representative measurement of a motion of the foot of the person, and
a processing unit adapted to receive the force and/or pressure measurement and the motion measurement, to compute at least one walking parameter and to control the emission of a stimulation at a walking moment according to the value of the walking parameter.

According to one embodiment, the insole additionally includes a stimulation unit adapted to be controlled by the processing unit and to emit the stimulation to stimulate the foot of the person,

According to another embodiment, the processing unit is adapted to determine in real time the walking moment and to control in real time the emission of the stimulation.

According to another embodiment, the processing unit is adapted to receive a new force and/or pressure measurement and a new motion measurement acquired after the stimulation has been emitted, and to modify at least one parameter of the stimulation.

According to another embodiment, the processing unit is adapted to receive a new force and/or pressure measurement and a new motion measurement acquired after the stimulation has been emitted, and to change the walking moment during which subsequent stimulation is emitted.

According to another embodiment, the processing unit is adapted to analyze a time series of the previously acquired and computed walking parameters over an hour, a day, a few days or over an even longer period, and to modify at least one parameter of the stimulation accordingly.

According to another embodiment, the processing unit is adapted to analyze a time series of the previously acquired and computed walking parameters over an hour, a day, a few days or over an even longer period, and for the walking moment during which subsequent stimulation is emitted.

According to another embodiment, the stimulation unit is adapted to emit an electrical stimulation.

According to another embodiment, the stimulation unit is adapted to emit a haptic stimulation.

According to another embodiment, the force and/or pressure measurement unit comprises capacitive sensors, each sensor comprising an upper electrode and a lower electrode, separated by a dielectric layer.

According to another embodiment, the processing unit is adapted to implement a learning operation to determine the parameters of the stimulation to be emitted by the stimulation unit.

According to another embodiment, the insole includes a communication module with an external server, controlled by the processing unit, and adapted to transfer the force and/or pressure measurement and the motion measurement stored to the external server, especially after the walking period of the person.

According to another embodiment, the insole is autonomous, the processing unit being adapted to control the emission of a stimulation without communicating with an external server, in particular without communicating with an external server over a period of several hours, preferably several days, preferably at least seven days.

According to another embodiment, the stimulation unit comprises a plurality of stimulation elements distributed over an upper surface of the insole.

According to another embodiment, the processing unit is adapted to determine an activity of the person, and to control the emission of a stimulation only if the person performs said activity.

The invention also relates to a system comprising an insole according to the invention and a stimulation unit separated from the insole.

The invention also relates to a stimulation method in which an insole is arranged in a shoe worn by a person, in particular during a walking period of the person, the method implementing the following steps:
a) acquire at least one measure representative of the force and/or pressure exerted on the insole by the person,
b) acquire at least one motion measurement of the foot of the person,
c) compute a walking parameter and determine a walking moment based on the force and/or pressure measurement and the motion measurement, and
d) emit a stimulation to stimulate the person at the walking moment.

According to one embodiment, the determination of the walking moment and the emission of stimulation are carried out in real time.

According to another embodiment, the stimulation method further comprises the following steps:
e) reception of a new force and/or pressure measurement and a new motion measurement acquired after the stimulation of step d) has been emitted,
f) modification of at least one parameter of the stimulation, and
g) repetition of the steps of the stimulation method.

### BRIEF DESCRIPTION OF THE DRAWINGS

Other characteristics, details and advantages of the invention will appear from the reading of the detailed description below, and from the analysis of the annexed drawings, on which:

FIG. 1 shows a front view of a pair of shoes, each shoe comprising an insole according to an embodiment of the invention.

FIG. 2A shows a perspective view from above of an insole according to an embodiment of the invention.

FIG. 2B shows a perspective view from below of an insole according to an embodiment of the invention.

FIG. 3 shows an exploded view of the insole of FIG. 2A.

FIG. 4 shows a synoptic diagram of an insole according to an embodiment of the invention.

FIG. 5 shows a diagram of the stimulation method according to an embodiment of the invention.

### DETAILED DESCRIPTION

The drawings and description below contain essentially elements of a definite character. They can therefore not only serve to better understand the present invention, but also contribute to its definition, if necessary.

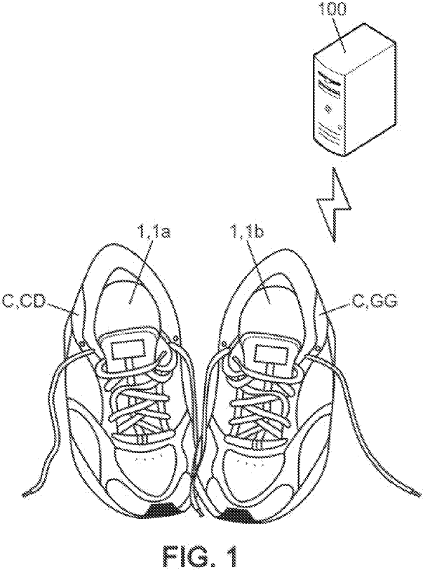

Referring first of all to FIG. 1, the invention has, as its first object, an insole 1 intended to be worn by a person.

The insole 1 is suitable to be inserted into a shoe C of the person.

The insole 1 can also be permanently integrated into shoe C, for example when manufacturing the shoe C, as being part of the sole of shoe C for example.

Shoe C can take many forms, such as a street shoe, a sports shoe or an orthopedic shoe, this list being not exhaustive.

In particular, FIG. 1 shows a first insole 1a of shoe, intended for example to be inserted into a right shoe CD, and a second insole 1b of shoe, intended for example to be inserted into a left shoe CG.

The first insole 1a of shoe can also be permanently integrated into the right shoe CD, and the second insole 1b of shoe can be permanently integrated into the left CG, for example when manufacturing said right shoe CD and left shoe CG, as being part of the sole of the shoes for example.

The first insole 1a and the second insole 1b are substantially similar, with the exception of transverse symmetry, and a single insole 1 will therefore be described below, its characteristics being shared by the first insole 1a and the second insole 1b.

In some embodiments, however, some characteristics may be reversed between the first and second insoles 1a, 1b.

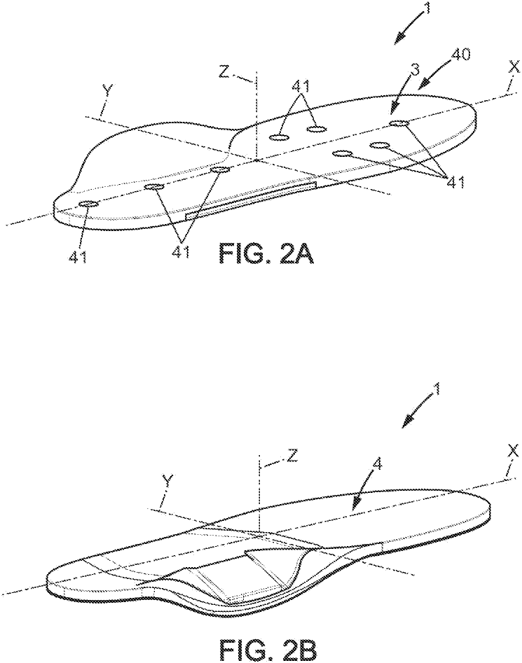

As further illustrated in FIG. 2A and FIG. 2B, insole 1 is substantially flat and extends in a horizontal plane X, Y, perpendicular to a direction of thickness Z.

“Substantially flat” means that insole 1 extends substantially in a plane, having large dimensions along a longitudinal direction X and a transverse direction Y (the transverse direction Y being perpendicular to the longitudinal direction X), and a relatively smaller dimension along a direction of thickness Z that is perpendicular to the longitudinal and transverse directions.

The insole 1 has a length or size according to the longitudinal direction X, greater than a width or size according to the transverse direction Y.

For example, the length of the insole 1 is at least twice its width.

Finally, the insole 1 has a thickness or clutter according to the direction of thickness Z, small compared to both its length and width.

For example, the thickness of insole 1 is at least ten times smaller than its length.

The insole 1 can thus have, for example, a thickness of less than one centimeter, preferably less than 0.75 centimeters, for example about 0.5 centimeters.

It should be noted that the insole 1 can have structures, bumps and small curves and therefore depart from a perfect plan. However, the extension of these structures, bumps and curves is understood to be small compared to the extension of insole 1 in the longitudinal direction X and the transverse direction Y.

The insole 1 extends between an upper surface 3, and a lower surface 4.

For example, the upper surface 3 is adapted to be in contact with a foot of a person hosted in the shoe C.

“The upper surface is in contact with a foot of a person” means that the foot of the person, which may be surrounded by an appropriate undergarment such as a sock, is in intimate contact, without intermediary, with the upper surface 3 of the insole 1.

The lower surface 4 of the insole 1 is also adapted to be in contact with a sole of the shoe C.

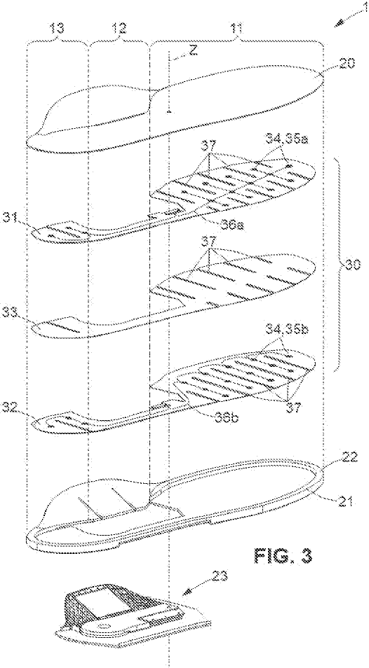

As shown in FIG. 3, the insole 1 comprises a frontal portion 11 arranged to come into contact with a front part of the foot, a middle portion 12 arranged to come into contact with a central part of the foot, for example a foot arch, and a rear portion 13 arranged to come into contact with a rear part of the foot.

The frontal portion 11, the middle portion 12 and the rear portion 13 are connected together to form a single element that can be more or less flexible.

In particular, the frontal portion 11 may extend over a width greater than a width of the middle portion 12 according to the transverse direction Y.

As shown in FIG. 3, the insole 1 is a multilayer element, e.g., laminated or comprising one or more layers embedded in a material chosen for example from polyurethane, ethylene vinyl acetate (EVA), thermoplastic polyurethane (TPU), thermoplastic rubber or silicone material.

The insole 1 includes, for example, an upper layer 20 which forms, in particular, the upper surface 3, and a lower layer 21 which forms, in particular, the lower surface 4. The upper layer 20 and the lower layer 21 can be welded together, especially on a perimeter 22 of the insole 2.

Although six layers are illustrated in FIG. 3, it is understood that the insole 1 may include a greater or lower number of layers.

In addition, the insole 1 may include a case 23. As shown in FIG. 3, case 23 forms the lower surface 4 with the bottom layer 21.

The insole 1 comprises a force and/or pressure measurement unit 30, a motion measurement acquisition unit 50 and a processing unit 60.

The insole 1 can also advantageously comprise a stimulation unit 40. Alternatively, stimulation unit 40 may be distinct from the insole 1, as discussed below.

In the embodiment of the invention illustrated in FIG. 2A, FIG. 2B and FIG. 3, the force and/or pressure measurement unit 30 and the stimulation unit 40 are located in the frontal portion 11 and in the rear portion 13 of the insole 1. Motion measurement acquisition unit 50 and processing unit 60 are located in the middle portion 12 of the insole 1.

However, this embodiment is illustrative and non-limiting, the force and/or pressure measurement unit 30, the stimulation unit 40, the motion measurement acquisition unit 50 and the processing unit 60 can be arranged differently in the insole 1.

### Force and/or Pressure Measurement Unit

Referring to FIG. 3, the force and/or pressure measurement unit 30 of the insole 1 is now described in more detail.

The force and/or pressure measurement unit 30 includes a plurality of sensors 34. The sensors 34 are located in front portion 11, middle portion 12 and/or rear portion 13.

It is thus possible to measure a pressure distribution, or a quantitative value of the force or pressure exerted on the foot, at the level of one or more regions of the sole of the person’s foot.

According to an example, it is thus possible to determine the position of the center of gravity of the person. In addition, by measuring the force and/or pressure over time, it is possible to obtain dynamic information about the force exerted by the person on the insole 1.

The sensors 34 are suitable for measuring pressure, tensile force, compressive force and/or shear force. The sensors 34 are advantageously capacitive, and include in particular an acquisition circuit (capacimeter type; not illustrated) to acquire a capacity value. But the sensors 34 can also be resistive or piezoelectric, or other.

According to the illustrated embodiment where the sensors 34 are capacitive, the force and/or pressure measurement unit 30 comprises a flexible upper layer 31 and a flexible lower layer 32. Both the upper layer 31 and the lower layer 32 extend globally along the longitudinal and transverse directions X, Y. The top layer 31 and the bottom layer 32 therefore face each other in the direction of thickness Z.

The force and/or pressure measurement unit 30 also includes a dielectric layer 33. The dielectric layer 33 is arranged between the upper layer 31 and the lower layer 32. The dielectric layer 33 is advantageously a flexible insulating layer as it will be detailed below.

Referring specifically to FIG. 3, each sensor 34 comprises an upper electrode 35a on the upper layer 31 and a lower electrode 35b on the lower layer 32. The upper electrode 35a and the lower electrode 35b extend perpendicular to the direction of thickness Z and face each other respectively in the direction of thickness Z.

For example, the top electrode 35a and the bottom electrode 35b can be squares of about 5 mm side, or can be discs of a few millimeters in diameter.

The upper electrode 35a and the lower electrode 35b are separated from each other at least by the dielectric layer 33.

Upper conductors 36a are also provided on the upper layer 31. The upper 36a conductors are electrically connected to the upper 35a electrodes of the 34 sensors. Similarly, lower conductors 36b are provided on the lower part 32. The lower 36a conductors are electrically connected to the lower 35b electrodes of the 34 sensors.

In one embodiment of the invention, the upper conductors 36a and the lower conductors 36b can be arranged to connect together the sensors 34. The sensors 34 are advantageously distributed on the surface of the insole 1 in a matrix.

By “matrix” we mean that the 34 sensors are connected to each other so that they can be used with a limited number of inputs/outputs. For example, if the force and/or pressure measurement unit 30 includes nine sensors 34, three input signals and three output signals are sufficient to be able to use the nine sensors 34. The result is a force and/or pressure measurement unit 30 that is particularly simple to implement.

In another embodiment of the invention, the upper conductor 36a and lower conductor 36b may be spatially multiplexed, that is to say arranged to connect each sensor 34 separately from each other.

In the absence of tensile, compression or lateral shear loads, the value of the C capacitance of a sensor 34 may be determined as a function of the thickness L of the dielectric layer 33 at the location of the sensor 34, the surface S of the upper electrode 35a and the lower electrode 35b and the dielectric constant ε of the material between the upper and lower electrodes 35a, 35b, in particular the dielectric layer 33, by the following equation:

$$C = ε S / L$$

The dielectric layer 33 is advantageously made of a dielectric material that is elastically deformable under tensile loads, compression loads and lateral shear. Under the effect of a compression or tensile load, the thickness L of the dielectric layer 33 at the location of the sensor 34 is modified and the capacity C of the sensor 34 varies. Under the effect of lateral shear, the overlap between the upper and lower electrodes varies and the capacity C of the sensor 34 varies accordingly.

In addition, in order to increase the flexibility of the insole 1 while allowing proper operation of the force and/or pressure measurement unit 30, the upper layer 31, the lower layer 32, and/or the dielectric layer 33 may include openings 37.

Such openings in a sole are for example described in the patent application EP 3235428.

According to the embodiment of FIG. 3, the openings 37 extend rectilinearly according to the transverse direction Y. However, openings 37 may have other forms or orientations.

In one embodiment of the invention, the sensors 34 are adapted to carry out a pretreatment of force and/or pressure measurements, for example at least one of the following pre-processings:
frequency filtering, for example frequency filtering in a range of temporal frequencies of interest,
a conversion of a unit of measurement capable, for example, of converting into a basic unit of the international system, and/or
sampling of measurements capable, for example, of sampling measurements with a sampling rate.

The processing unit 60 receives measurements from the sensors 34, possibly pre-processed as detailed above.

If the force and/or pressure measurements received are not pre-processed, the processing unit 60 may in particular implement one and/or other of the pre-processings detailed above.

### Stimulation Unit

The stimulation unit 40 is adapted to emit stimulation.

Stimulation can be emitted to the person’s leg or foot. The stimulation can act in particular at the level of the sole of the foot of the person, more particularly at the level of the arch, the heel or the metatarsal heads of the foot.

However, according to another embodiment, the stimulation can also be emitted at the level of another part of the person’s body, such as the anterior tibial nerve, the peroneal nerve, the median nerve of the arm, the spine, the abdomen, the neck, the twin muscle, the hand, the ankle, the shoulder, the hamstring, the quadriceps, the lower back, or the tip of an amputated limb.

The stimulation thus allows to act on the muscular contraction, on the blood circulation or the cerebral functioning of the person.

For this purpose, the stimulation unit may be separated from the insole 1. The stimulation unit is then placed against or near the part of the body to be stimulated.

According to the embodiment illustrated, the stimulation unit 40 is more particularly adapted to emit stimulation at the level of the sole of the foot of the person.

The insole 1 then advantageously comprises the stimulation unit.

For this purpose, as illustrated in FIG. 2A, the stimulation unit 40 comprises a plurality of stimulation elements 41. The stimulation elements 41 are located in the frontal portion 11, the middle portion 12 and/or the rear portion 13. The stimulation elements 41 can be distributed in a matrix (this term should be understood as before).

Stimulation elements 41 are located on the upper surface 3 of the insole 1. The stimulation elements 41 are advantageously distributed on the surface of the insole 1.

It is thus possible to stimulate one or more regions of the sole of the person’s foot selectively or simultaneously.

According to a first embodiment, the stimulation elements 41 are adapted to emit electrical stimulation.

According to one embodiment, the stimulation elements 41 are adapted to emit a Transcutaneous Electrical Nerve Stimulation (TENS).

The stimulation emitted can thus be emitted in the form of a signal, in particular electrical, defined by one or more parameters of the stimulation. These parameters can be chosen for example from the shape of the emitted signal (e.g., sinusoidal, rectangular, triangular, or other), the amplitude and the frequency or frequencies of the wave. For example, the signal can be an impulse.

Furthermore, a stimulation may also consist of a plurality of signals emitted successively one after the other, for example in the form of a signal train, in which case a parameter of the stimulation may also be the frequency of repetition of the signal during the same stimulation.

In the embodiment where the stimulation is electrical, the current emitted by the stimulation elements 41 may for example be of low amperage, for example of an intensity between 10 mA (milliamperes) and 30 mA.

The stimulation elements 41 can emit pulsations whose frequency is between 40 Hz (Hertz - pulsation per second) and 150 Hz.

According to another embodiment, the stimulation elements 41 are adapted to emit haptic or vibrational stimulation. The stimulation elements 41 can then be electromechanical devices comprising an electromagnet and a vibrating element.

According to another embodiment, the stimulation elements 41 are adapted to emit visual stimulation. The stimulation elements 41 can then be one or more light sources.

According to another embodiment, the stimulation elements 41 are adapted to emit sound stimulation. The stimulation elements 41 can then be one or more speakers.

### Motion Measurement Acquisition Unit

The motion measurement acquisition unit 50 is adapted to acquire at least one measurement of a motion of the person, or even a plurality of motion measurements.

The motion measurement is advantageously an angular, speed or acceleration measurement.

An angular measurement may be used to adjust the intensity of stimulation. If the amplitude of rotation of the motion is insufficient, the intensity of the simulation can be increased.

An acceleration measurement can be used to quantify the impact speed during motion and adjust the frequency of the stimulation wave trains or the unit period of stimulation. If the measured impact velocity increases, it may be possible to increase the frequency of the stimulation wave trains or decrease the unit period of the stimulations. In addition, an acceleration measurement may be used to adjust the time moment of stimulation emission. For this purpose, the motion measurement acquisition unit 50 includes one or more accelerometers and/or one or more gyroscopes and/or one or more inclinometers (not shown) adapted to detect linear or angular accelerations and inclinations, at the level of the foot of the person.

The combination of the different measuring tools allows to improve the accuracy in the measurement of the rotational speed, in the measurement of the walking speed.

These elements of the motion measurement acquisition unit 50 can be arranged inside the case 23 of the insole 1.

### Processing Unit

The processing unit 60 of the insole 1 is now described in more detail.

The processing unit 60 may for example include on-board electronics or a processor (not shown) arranged inside case 23 of insole 1.

The force and/or pressure measurement unit 30, the stimulation unit 40, the motion measurement acquisition unit 50 and the processing unit 60 are functionally connected to each other, in particular electrically. In particular, the processing unit 60 is electrically connected to the sensors 34 and the stimulation elements 41.

Thus, the processing unit 60 is suitable for controlling and receiving information from the force and/or pressure measurement unit 30 and the motion measurement acquisition unit 50, and is also suitable for commanding and controlling the emission of a stimulation by the stimulation unit 40.

In the embodiment where the insole 1 comprises the stimulation unit 40, the communication between the force and/or pressure measurement unit 30, the stimulation unit 40, the motion measurement acquisition unit 50 and the processing unit 60 is particularly fast and high flow since they are all arranged in the insole 1, and therefore relatively close to each other.

Preferably, the pressure measurement and the motion measurement (accelerometers, gyroscopes) are performed on the same printed circuit board allowing a segmentation of the steps and a calculation of the spatio-temporal parameters of walking in real time.

The processing unit 60 is adapted, in real time, to receive one or more measurements of the force and/or pressure measurement unit 30 and one or more motion measurements of the motion measurement acquisition unit 50, to compute at least one, or even several, walking parameters (stride length/width, stride speed, ground contact time, flight time, single support time, double support time, displacement of the pressure center) and to control the emission of a stimulation by the stimulation unit 40. The processing unit 60 is suitable to receive one or more measurements of the force and/or pressure measurement unit 30 and one or more motion measurements of the motion measurement acquisition unit 50 and one or more measurements of the force and/or pressure measurement unit 30 of the opposite sole and one or more motion measurements of the motion measurement acquisition unit 50 of the opposite sole to compute combined parameters.

The walking parameter allows to characterize the motion of the person. Thus, the walking parameter can indicate whether it is necessary, and if so to what extent, to stimulate the person. For example, we can identify if the person is in an active phase of walking and thus stop the stimulation. For example, if the person’s walking speed increases, it will be possible to increase the frequency of the wave trains or decrease their period.

Walking is a cyclical motion in which easily recognizable events are repeated.

A step is thus defined each time a leg of the person propels itself forward.

A walking cycle begins with the initial contact of one foot and ends with the next contact of the same foot, which in turn is the initial contact of the next walking cycle.

The walking cycle can thus be divided into a stance phase, in which one foot is in contact with the ground, and a swing phase, in which the same foot, in the air, moves forward. The stance phase consists of double support phases, in which both feet of the person are in contact with the ground and single support phases in which only one foot is in contact with the ground.

For a normal running cycle, the stance phase represents, on average, 60% of the cycle compared to 40% for the swing phase. These two phases are delimited by the toe off (around 60%) and by the two contacts of one of the heels defining the beginning (0%) and the end of the walking cycle (100%).

The walking parameters include spatio-temporal parameters and angular parameters to characterize the motion of the person.

A spatial parameter can be chosen from step length, stride length, pitch angle, pitch width (distance or angle), stride width or pitch height. It is also possible to determine the position of the center of gravity of the pressure for each foot of the person or his trajectory for example.

A temporal parameter can be chosen from the person’s cadence (number of steps per minute), the walking speed, the double support time, the single support time (duration of the stance phase in which only one foot is in contact with the ground), the asymmetry of the parameters (difference between the two limbs) of a limb likely to be stimulated or of the other limb.

An angular parameter can be chosen from a relative joint motion between members of the person causing the walk, such as the ankle. Joint motions can vary in particular in the sagittal, frontal or transverse plane of the person.

The gait parameters indicated above are not limiting and other parameters are possible or include a combination of the spatio-temporal and angular parameters above.

For example, if the force and/or pressure measurement unit 30 measures a strong pressure exerted at the heel of the foot of the person, and the motion measurement acquisition unit 50 measures a sudden deceleration, it can be considered that the person passes from a swing phase to a stance phase by putting his foot on the ground and that one is at the beginning or at the end of the walking cycle. It can then be finally measured the length of steps of the person for example.

From the calculation of the walking parameter, the emission of the stimulation by the stimulation unit 40 can thus be synchronized with a walking moment.

By “synchronized with a walking moment”, we mean in particular that the stimulation emitted by the stimulation unit 40 is synchronized in time with a specific moment of the walking cycle. A walking moment can for example be the moment when a foot of the person comes into contact with the ground, or the moment when the foot is in the swing phase or the moment of the phase of double support. For example, one can initiate the stimulation of the anterior tibial at the time of toe-off or stop the stimulation at the time of the heel strike. In a finer way, one can gradually increase the intensity of the stimulation in anticipation of the detachment of the toes.

It is thus possible to emit the stimulation at a time when it is likely to be most effective and to have a satisfactory effect on the person. For example, the emission of stimulation at the exact moment of the heel strike or just before the toe-off will improve the fluidity of walking. For example, the emission of the stimulation at the time of the double support phase will indicate which foot to be lifted as a priority.

By “real time” is meant an implementation of the stimulation method such that the processing unit 60 can determine a walking moment and control the emission of a stimulation synchronized with this walking moment and according to the parameters of the stimulation.

From the computation of the walking parameter, the stimulation parameters by the stimulation unit 40 can also be modified.

It is thus possible to emit an adapted and sufficient stimulation, likely to be the most effective and to have a satisfactory effect on the person.

### Stimulation Method

It is known that changes in walking parameters such as speed, pitch height, length and frequency of steps in a person are signs of impairment or degradation of walking abilities or quality.

These difficulties may appear due to a deficiency, paralysis, or abnormal position of the foot.

Mobility disorders can include muscle fatigue, spasticity, freezing of gait, foot drop, loss of balance, asymmetry of gait (between the two lower limbs), venous insufficiency, overactive bladder, phantom limb pain.

Mobility disorders are often associated with certain pathologies or problems related to the person more general, such as Parkinson’s disease, the occurrence of a stroke, multiple sclerosis, the age of the subject, obesity, etc.

The processing unit 60 and stimulation unit 40 are then adapted to implement a person stimulation method, which will now be described in more detail.

The implementation of the stimulation method according to the invention may not be limited to the walking of the person but can also be carried out during other motions on foot, for people with prostheses on one of the lower limbs for example during a run for example, or even during other types of activities that require a pressure effort at the level of the arch foot of the person (by bike by example).

To implement the stimulation method, the processing unit 60 can, from the acquisition of force and/or pressure measurements, motion measurements, and the calculation of the walking parameter, determine the walking moment.

For example, the walking parameter may indicate that the person’s foot comes into contact with the ground by exerting a higher pressure force on one side of the foot, to the detriment of the other side of the foot. In addition, the walking parameter can also indicate an unbalanced foot tilt.

In another example, the walking parameter may indicate that the person’s pitch is abnormally small.

The processing unit 60 is then adapted to control the stimulation unit 40 so that a stimulation is emitted in synchronized with the walking moment.

Thus, a stimulation can for example be emitted when the foot of the person comes into contact with the ground, in order to restore the balance of the person and to ensure that it exerts an equal force of pressure on both sides of the foot.

According to another example, a stimulation can be emitted in order to force the person to take a step of greater length.

For this purpose, the stimulation can be emitted by only certain stimulation elements 41 in order to stimulate certain regions of the foot only, for example the frontal portion 11, the middle portion 12 and/or the rear portion 13, or an inner and/or outer side of the foot or a sequence of these areas depending on the unfolding of the step.

The processing unit 60 is also adapted to control the stimulation unit 40 so that a stimulation is emitted with satisfactory simulation parameters.

After or in parallel with the emission of the stimulation, the processing unit 60 is adapted to acquire again the force and/or pressure measurements and the motion measurement(s).

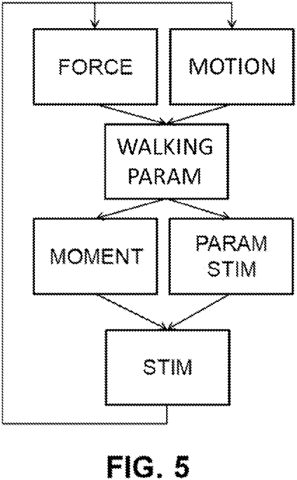

The stimulation method thus works in a closed loop, as shown in FIG. 5.

It can thus be assessed whether the stimulation has had an effect on the person from the force and/or pressure measurements and the motion measurements newly acquired by the systems of one or both lower limbs. In particular, it can be assessed whether the person’s ability to move has been satisfactorily altered by the emission of stimulation.

Depending on the force and/or pressure measurements and newly acquired motion measurements, the processing unit 60 can be adapted to change the time of walking or adapt the parameters of stimulation.

For example, if it appears that the stimulation has had a beneficial effect, but that it is small, it may be decided to increase the intensity of the stimulation in order to improve the effectiveness of the next stimulations that will be emitted.

According to another example, if it appears that the stimulation was emitted in a synchronized way with a walking moment too late or poorly selected, it may be decided to advance the moment when the stimulation must be emitted, especially during the next walking cycle, in order to improve its effectiveness.

The stimulation is then repeated advantageously several times during walking, especially during a walking period of the person.

A stimulation method is then issued periodically, or almost periodically, during a period of motion of the person. For example, stimulation can be emitted at each step or cycle of walking a person during a given period.

Since the stimulation parameters and/or the choice of the walking moment are likely to evolve all along the emission of the stimulations, it is thus possible to implement a reinforcement learning operation to refine the stimulation parameters, for example the amplitude, the shape of the stimulation signal, or others.

For example, if the efficiency of the stimulation is reduced, it may for example change the frequency of the wave trains or the intensity of simulation by reducing or increasing the unit period of the stimulations, or the number of stimulations in the wave train, or the time between two stimulations in a wave train, or the period between two wave trains; or by shifting the time for the implementation of post-stimulation stimulation.

For example, it will be possible to study the walking parameters over a fairly long period of time in order to identify trends in the improvement of these parameters. If the parameters no longer evolve or evolve too slowly, it is possible to increase the amplitude of stimulation or change the type of wave train or its frequency.

For example, it will be possible to vary different stimulation parameters (frequency and/or intensity), observe the effect and select the most effective parameters for the given patient.

For example, it will be possible to study the moment of stimulation during a number of walking cycles, identify the best moment of stimulation and adapt the stimulation parameters.

Thus, the processing unit 60 can be adapted to control a stimulation according to the values of walking parameters, and/or the values of force and/or pressure measurements and motion measurements previously acquired, thus taking into account the entire history of measurements and values acquired during previous stimulations concerning the motion of the person.

For this purpose, the processing unit 60 can implement an artificial intelligence algorithm, for example a neural network.

Such a reinforcement learning operation is particularly appropriate since the force and/or pressure measurements, or the motion measurements, are highly variable from one person to another. In addition, it allows to take into account the phenomenon of habituation to stimulation in the same person over time, in order to ensure that the stimulation method continues to be effective over time and in different environments.

The stimulation parameters can thus be adjusted during the implementation of the stimulation method, for example to adjust the said parameters to the person.

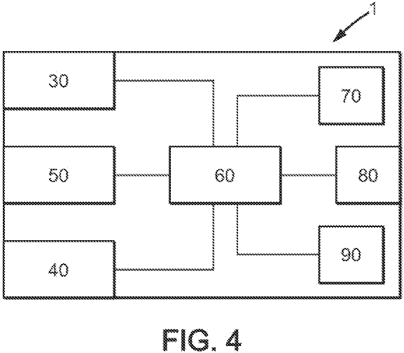

As shown in FIG. 4, the standalone sole 1 may include a memory of 70. The memory 70 is adapted to be mounted on the autonomous sole 1, for example in the case 23. The memory 70 can be mounted permanently or can be a removable module, for example a memory card such as an SD card (“Secure Digital”).

In particular, the memory 70 is functionally connected to the processing unit 60. The memory 70 can be controlled by the processing unit 60 in such a way as to record the force and/or pressure measurements, the motion measurements, the stimulation parameters and/or the walking moment, over a period of several days, for example at least seven days so as to cover a week of a person for use in autonomy.

In addition, as shown in FIG. 4, the standalone sole 1 can also include a communication module 80 with an external server 100, shown in FIG. 1. The communication module 80 can be mounted on the standalone sole 1 and controlled by the processing unit 60.

In particular, the processing unit 60 can be adapted to control the communication module 80 to transfer the force and/or pressure measurements, motion measurements, stimulation parameters and/or walking moment stored in the memory 70, to the external server 100 or to the sole of the opposite limb. This transfer operation may in particular be implemented after a period of walking of the person.

The communication module 80 can advantageously be a wireless communication module, for example a module implementing a protocol such as Bluetooth, Wi-Fi, SIGFOX or LoRa technology.

In this way, when the person is in a walking period, he is not hindered by cables, especially if it is necessary to carry out data transmissions during the period of motion on foot.

In addition, as shown in FIG. 4, the insole 1 may include at least one battery 90. The battery 90 can advantageously be flexible. The battery 90 can be recharged by wireless induction.

The battery 90 stores electrical energy and can be adapted in particular to power the force and/or pressure measuring unit 30, the stimulation unit 40 and the processing unit 60, as well as, if necessary, the memory 70 and the communication module 80. The battery 90 is preferably suitable to provide power over a period of several days without recharging.

In this way, the insole 1 can operate autonomously during a walking period of the person. Thus, the insole 1 is autonomous and adapted to implement one or more stimulation methods without communicating with the external server 100, in particular without communicating with the external server 100 over a period of several days, preferably at least seven days with for example a detection of the phases of inactivity.

By “autonomous”, it is thus meant that the insole 1 can operate for an extended period, preferably several days, in particular at least seven days, without the need to be recharged with electrical energy, to communicate with external elements such as the outdoor server 100 or to be structurally connected to an external device.

In this way, the insole 1 is suitable to be used in the daily life of the person without imposing special constraints. In addition, the insole includes all the necessary elements to implement the stimulation method as described above, and can therefore be easily implemented.

Of course, the invention is not limited to the embodiments described above and provided only as an example. It includes various modifications, alternative forms and other variants that may be considered by the skilled person in the context of the present invention and in particular all combinations of the different modes of operation described above, which can be taken separately or in combination.

1-17. (canceled)

18. An insole configured to be disposed in a shoe worn by a person, in particular during a period of walking of the person, the insole comprising:
a force and/or pressure measurement unit configured to acquire at least one measurement representative of the force and/or pressure exerted on the insole by the person,
a motion measurement acquisition unit configured to acquire at least one measurement representative of a motion of the person’s foot, and
a processing unit configured to receive the force and/or pressure measurement and the motion measurement, to compute at least one walking parameter and to control the emission of a stimulation at a walking moment according to the value of the walking parameter.

19. The insole according to claim 18, further comprising a stimulation unit configured to be controlled by the processing unit and to emit stimulation to stimulate the person’s foot,.

20. The insole according to claim 18, wherein the processing unit is configured to determine in real time the walking moment and to control in real time the emission of stimulation.

21. The insole according to claim 18, wherein the processing unit is configured to receive a new force and/or pressure measurement and a new motion measurement acquired after the stimulation has been emitted, and to modify at least one stimulation parameter.

22. The insole according to claim 18, wherein the processing unit is configured to receive a new force and/or pressure measurement and a new motion measurement acquired after the stimulation has been emitted, and to modify the walking moment during which a subsequent stimulation is emitted.

23. The insole according to claim 19, wherein the stimulation unit is configured to emit electrical stimulation.

24. The insole according to claim 19, wherein the stimulation unit is configured to emit haptic stimulation.

25. The insole according to claim 18, wherein the force and/or pressure measurement unit comprises capacitive sensors, each sensor comprising an upper electrode and a lower electrode, separated from each other by a dielectric layer.

26. The insole according to claim 18, wherein the processing unit is configured to implement a learning operation of the parameters of the stimulation emitted.

27. The insole according to claim 18, comprising a communication module with an external server, controlled by the processing unit, and adapted to transfer the force and/or pressure measurement and the motion measurement stored to the external server, in particular after a walking period of the person.

28. The insole according to claim 27, wherein the insole is autonomous, the processing unit being adapted to control the emission of a stimulation without communicating with an external server, in particular without communicating with the external server over a period of several hours.

29. The insole according to claim 19, wherein the stimulation unit comprises a plurality of stimulation elements distributed on a superior surface of the insole.

30. The insole according to claim 18, wherein the processing unit is adapted to determine an activity of the person, and to control the emission of stimulation only if the person performs said activity.

31. A system comprising an insole according to claim 18, and a stimulation unit distinct from the insole.

32. A stimulation method in which an insole is disposed in a shoe worn by a person, in particular during a period of walking of the person, the process implementing the following steps:
a) acquiring at least one measure representative of the force and/or pressure exerted on the insole by the person,
b) acquiring at least one measurement representative of a motion of the person’s foot,
c) computing a walking parameter and determining a walking moment based on the force and/or pressure measurement and the motion measurement, and
d) emitting stimulation to stimulate the person at the walking moment.

33. The stimulation method according to claim 32, wherein the determination of the walking moment and the emission of stimulation are carried out in real time.

34. The stimulation method according to claim 32, further comprising the following steps:
e) receiving a new of force and/or pressure measurement and a new motion measurement acquired after the stimulation of step d) has been emitted,
f) modification of at least one parameter of the stimulation, and
g) repetition of steps a) to d).
